(19) United States
(12) Patent Application Publication
(10) Pub. No.: US 2021/0169632 A1
(43) Pub. Date: Jun. 10, 2021

(54) METHODS AND DEVICES FOR TRANS-BRONCHIAL AIRWAY BYPASS

(71) Applicant: EOLO MEDICAL INC., New York, NY (US)

(72) Inventors: Qi LI, Jersey City, NJ (US); Don TANAKA, Saratoga, CA (US); Lilip LAU, Los Altos, CA (US); Mark GELFAND, New York, NY (US)

(21) Appl. No.: 16/768,815

(22) PCT Filed: Nov. 30, 2018
(86) PCT No.: PCT/US2018/063256

§ 371 Date: Jun. 1, 2020

Related U.S. Application Data
(60) Provisional application No. 62/593,683, filed on Dec. 1, 2017.

(51) Int. Cl. A61F 2/04; A61B 17/3207; A61B 1/267; A61F 2/962; A61F 2/88; A61B 1/012

(52) U.S. Cl. CPC A61F 2/04; A61B 17/320725; A61B 1/2676; A61B 2017/242; A61F 2/88; A61B 1/012; A61F 2002/043; A61F 2/962

(57) ABSTRACT

Endobronchial methods, devices and systems for treating emphysema by creating an airway bypass in a patient's airway including a longitudinal fenestration held open by an airway expander.

### TECHNICAL FIELD

The present disclosure is directed generally to methods and devices for trans-bronchial airway bypass for treatment of Chronic Obstructive Pulmonary Disease (COPD), including emphysema. In particular endobronchial systems for creating an airway bypass in a patient's airway and delivering a device for maintaining patency of the bypass, devices for maintaining patency of the bypass, methods of using the systems and devices, and methods of treating a hyperinflated lung (e.g., COPD) are disclosed herein.

### BACKGROUND

Airway bypass by transbronchial fenestration has been shown to improve forced expiratory volume and flow in explanted emphysematous human lungs. For example, Exhale® is a drug-eluting stent (Broncus Technologies, Mountain View, Calif.) used in homogeneous emphysema subjects with severe hyperinflation. These stents traversed the airway wall and created an air passage from parenchyma into the airway. The passage was created by a puncture, dilated by balloon, and reinforced by a stent that crossed the wall of the airway. However, the following problems were encountered: only proximal (e.g., generations 3 to 4) airways were treated because they were accessible under direct vision using a standard bronchoscope (this position is not optimal for bypass and creates risks of perforating a large blood vessel); stents were not well secured in airway walls and patients often coughed them out; stent openings creating the artificial passageways were very small and within months closed by tissue growth, secretions and mucus plugs; once closed stents could not be re-opened or removed.

Similarly, US20050066974 assigned to Pulmonx discloses achieving a desired fluid flow dynamic to a lung region by deploying various combinations of flow control devices and bronchial isolation devices in one or more bronchial passageways that communicate with the lung region. The flow control devices are implanted in channels that are formed in the walls of bronchial passageways of the lung. The flow control devices regulate fluid flow through the channels. The bronchial isolation devices are implanted in lumens of bronchial passageways that communicate with the lung region in order to regulate fluid flow to and from the lung region through the bronchial passageways.

In another example, cutting longitudinal fenestrations in the airways to perform airway bypass is known but such fenestrations have natural tendency to close and heal. Thus there is a need for improved airway bypass methods and devices.

### SUMMARY

An invention has been conceived and is disclosed herein that is related to methods, devices, and systems for treating a patient with emphysema by reducing volume of a hyper-inflated portion of the patient's lung comprising: creating an airway bypass passage in a wall of a target location in an airway, deploying an airway expander, such as a stent or helical wire, in the target location in the airway, the airway expander configured to expand the airway bypass passage by expanding the airway diameter. Optionally, the step of creating an airway bypass passage comprises delivering an airway cutting device through the patient's airway to the target location and cutting a fenestration. The delivering step may comprise delivering through a bronchoscope or a sheath or over a guidewire.

The airway-cutting device may be a deployable blade deployed by a balloon, a pull wire, retracting a sheath, or a stent.

The airway bypass passage may be a fenestration with a length in a range of 2 to 25 mm.

The airway expander may be placed longitudinally in the airway.

The airway expander, at a maximum open, state may have an outer diameter greater than the target location in the airway, optionally in a range of 1.5 to 2.5 times greater, optionally 1 to 10 mm greater. The airway expander in its minimum closed state has an outer diameter in a range of 1 to 3 mm. The airway expander may comprise a middle region having a circumference that is larger than the circumference of the airway in it's expanded state, optionally 1 to 5 mm larger, 1 to 3 mm larger, or 10% to 30% of the circumference of the airway.

The airway expander may comprise a one-way valve that allows air to expel from the airway bypass passage but not enter it. The one-way valve may comprise a membrane.

The airway expander may comprise a one-way valve that allows air to expel from the airway but not enter it.

The airway expander may be a dual channel structure. One of the channels, Channel 1, faces the cutting slit or the airway bypass. The other channel, Channel 2, is on the opposite side or the further side to the airway bypass. Channel 1 may comprise at least one one-way valve that allows air to expel from the airway bypass but not enter it. Channel 2 may be a normal channel with complete patency. The two channels may be divided by a stent covered by a membrane, or divided by a membrane directly.

In some embodiments a method treatment further comprises a step of expanding the airway expander a repeated time, optionally comprising delivering a dilating balloon to the airway expander and inflating the dilating balloon to apply pressure to the inside of the airway expander.

The target location for creating a fenestration and implanting an expander may be a generation 4 or higher airway and may be adjacent emphysematous lung parenchyma or adjacent a hyperinflated portion of lung parenchyma.

In some embodiments the airway expander and the cutting device are the same device. The step of making the airway bypass passage may be accomplished during the step of deploying the airway expander.

In one embodiment, the invention is a system of treating emphysema by creating an airway bypass comprising a cutting device configured to create a longitudinal cut in a patient's airway and an airway expander configured to allow air to pass through the longitudinal cut in the wall of the airway and through the airway. The cutting device and the airway expander in their undeployed states can pass through a working channel of a bronchoscope, the working channel having an inner diameter in a range of 2 mm to 5 mm. The cutting device may be configured to deliver and deploy the airway expander. The cutting device may comprise a deployable balloon mounted to an elongate tubular structure and a blade mounted to the deployable balloon, wherein the blade is concealed when the deployable balloon is in an undeployed state and the blade is exposed when the deployable balloon is in a deployed state. The blade may comprise a height in a range of 0.25 mm to 2 mm and a length in a range of 2 to 15 mm. The cutting device comprises a deployable blade that is concealed in an undeployed state and is configured to pierce a wall of the airway and create a fenestration when in a deployed state.

In some embodiments the airway expander is a stent for dilating a portion of the airway and maintaining patency in the longitudinal cut in the airway comprising a proximal region, a middle region, and a distal region, wherein the middle region has a larger circumference than the proximal and distal regions when the stent is in its expanded state. The proximal and distal regions may have an outer diameter in a range of 1 to 5 mm and the middle region has an outer diameter in a range of 2 to 10 mm when the stent is in its expanded state and all regions have an outer diameter less then 3 mm when the stent is in its delivery state. The airway expander may be a deployable helical tube configured to be delivered through a lumen having an inner diameter in a range of 2 mm to 5 mm. The deployable helical tube may comprise a grasping protrusion to facilitate delivery or retrieval.

In another embodiment, the invention is an airway expander may be configured to be delivered to an airway having a natural diameter in a compressed state, to be positioned in the airway such that a longitudinal axis of the airway expander is oriented with a longitudinal axis of the airway, and to dilate the circumference of the airway in an expanded state beyond the natural diameter of the airway. The airway expander may be configured to dilate the airway more than 20% of the natural diameter. The airway expander may be configured to be expandable in radial increments of 10% to 30% and wherein each increment is expandable by a corresponding balloon dilator. The airway expander may have substantially open cells allowing passage of mucus. The airway expander may further comprise a graspable protrusion and is configured to be removed from the airway by pulling on the graspable protrusion. The airway expander may further comprise at least one valve configured to allow air to exit through the airway and resist air entering through the airway. The at least one valve may be positioned on a distal end of the airway expander or on a proximal end of the airway expander.

A method of reducing a volume of a target section of a lung by collapsing or partially collapsing the target section of the lung, the method comprising steps of: delivering within an airway leading to the target section an intra-bronchial valve device having an obstructing member supported on a support structure, the obstructing member being configured to preclude air from being inhaled into the target section, while allowing air to be exhaled from the target section, the valve device further comprising at least one airway expander configured to expand the airway diameter, the airway expander configured to bypass natural airways by creating and supporting a fenestrated air passage into lung parenchyma.

In some embodiments a method for lung volume reduction comprises steps of: creating an airway bypass passage between a targeted area of lung parenchyma adjacent to a targeted airway by opening a fenestration in the airway wall; implanting at least one airway expander configured to expand a diameter of the airway at the site of the airway bypass passage, further expanding the fenestration and preventing or at least delaying its closure.

Further aspects of the invention are discussed below:

According to a first aspect, the medical device assembly comprises: a cutting device (70, 85, 95) configured to create a cut in an airway passage (40, 41, 42) within a lung, wherein the cutting device has a collapsed mode in which the cutting device has a reduced outer dimension, and an expanded mode in which the outer dimension of the cutting device expands to at least an inner dimension of the airway passage to cut an opening (65) in the airway passage, and an airway expander (80, 105, 140, 150, 200, 300, 400, 500, 600, 700) configured to be positioned within the airway passage proximate (41) to the opening, wherein the airway expander has a collapsed mode in which the airway expander has a reduced outer dimension, and an expanded mode in which the outer dimension of the airway expander expands to displace radially outward the airway and to expand the opening formed in the airway, wherein the outer dimension of the airway expander in the expanded mode is larger than the outer dimension of the cutting device in the expanded mode.

According to a second aspect which incorporates the first aspect, the medical device assembly further includes a bronchoscope (10) with a working channel, wherein the reduced outer dimension of the cutting device is sufficiently small to pass through the working channel of the bronchoscope.

According to a third aspect which incorporates the first aspect and optionally the second aspect, The medical device assembly of claim 1 or 2 further comprising a bronchoscope (10) with a working channel, wherein the reduced outer dimension of the airway expander while in its respective collapsed mode is sufficiently small to pass through the working channel of the bronchoscope.

According to a fourth aspect that incorporates the first aspect and may incorporate the second and/or third aspect, the cutting device includes a cutting element (72, 88, 89, 96, 97) on an outer surface the cutting device, and the cutting element is configured to form the cut (65) in the airway passage while the cutting device is in the expanded mode.

According to a fifth aspect that incorporates the first aspect and may incorporate one or more of the second and fourth aspects, the airway expander is a stent or a helical wire (80, 105, 140, 150, 200, 300, 400, 500, 600, 700).

According to a sixth aspect that incorporates the first aspect and may incorporate one or more of the second to fifth aspects, the working channel of the bronchoscope has an inner diameter in a range of 2 mm to 5 mm, or less than 2 mm, or 1 mm to 6 mm, or 3 mm to 6 mm, or 4 mm to 8 mm.

According to a seventh aspect that incorporates the first aspect and may incorporate one or more of the second to eighth aspects, wherein the cutting device is configured to deliver and deploy the airway expander.

According to another aspect that incorporates the first aspect and optionally one or more of the other prior aspects, the cutting device includes a deployable balloon (71) mounted to an elongate tubular structure (70) and a cutting element (72, 88, 89, 96, 97) mounted to the deployable balloon.

According to another aspect that incorporates the first aspect and optionally one or more of the other prior aspects, the cutting device includes a blade (72, 88, 89, 96, 97) having a height in a range of one or more of 0.25 mm to 2 mm, less than 0.22 mm or 0.2 mm to 1.3 mm, and/or a length in a range of one or more of 2 to 15 mm, 4 mm to 12 mm, 7 mm to 17 mm, and 10 mm to 15 mm.

According to another aspect that incorporates the first aspect and optionally one or more of the other prior aspects, wherein the cutting device comprises a deployable blade (87, 88, 89, 90, 96, 97), which is optionally a deployable blade.

According to another aspect that incorporates the first aspect and optionally one or more of the other prior aspects, wherein the blade (72, 88, 89, 96, 97) of the cutting device has a height in a range of 0.25 mm to 2 mm.

According to another aspect that incorporates the first aspect and optionally one or more of the other prior aspects, wherein the blade (72, 88, 89, 96, 97) of the cutting device has and a length in a range of 2 to 15 mm.

According to another aspect that incorporates the first aspect and optionally one or more of the other prior aspects, wherein the blade (72, 88, 89, 96, 97) of the cutting device is a straight blade, optionally wherein the cutting device has a single straight blade.

According to another aspect that incorporates the first aspect and optionally one or more of the other prior aspects, wherein the blade of the cutting device comprises a deployable blade (87, 88, 89, 90, 96, 97).

According to another aspect that incorporates the first aspect and optionally one or more of the other prior aspects, wherein the cutting device comprises a spring and a delivery sheath (86) and wherein the spring is configured to act on the deployable blade (96) such that the deployable blade (96) is deployed by the spring when the delivery sheath (86) is retracted from the blade.

According to another aspect that incorporates the first aspect and optionally one or more of the other prior aspects, wherein the blade (87, 88, 89, 90, 96, 97) of the cutting device is elastically mounted on the cutting device.

According to another aspect that incorporates the first aspect and optionally one or more of the other prior aspects, wherein the airway expander includes a stent (80, 105, 140, 150, 200, 300, 400, 500, 600, 700) including a proximal region (107, 202, 302, 402, 502, 602, 702), a middle region (106, 201, 301, 401, 501, 601, 701), and a distal region (108, 203, 303, 403, 503, 603, 703), wherein the middle region has a larger circumference than the proximal and distal regions when the stent is in the expanded mode, and the middle region is configured to abut the opening (65) and the proximal and distal regions are configured to engage the airway respectively proximal and distal to the opening.

According to another aspect that incorporates the first aspect and optionally one or more of the other prior aspects, wherein the proximal and distal regions have an outer diameter in a range of 1 to 5 mm, 2 to 6 mm and/or 0.5 mm to 6 mm, and/or the middle region has an outer diameter in a range of 2 to 10 mm, 1 to 8 mm, and/or 4 to 12 mm when the stent is in its expanded mode, and all regions have an outer diameter less than 3 mm, less than 4 mm and/or less than 5 mm when the stent is in its delivery state.

According to another aspect that incorporates the first aspect and optionally one or more of the other prior aspects, wherein the airway expander includes a grasping protrusion (109, 121. 145, 204, 304, 404, 504, 604, 704).

According to another aspect that incorporates the first aspect and optionally one or more of the other prior aspects, wherein the airway expander has substantially open cells allowing passage of mucus.

According to another aspect that incorporates the first aspect and optionally one or more of the other prior aspects, wherein the airway expander further comprises at least one valve (141, 151, 206, 306, 313, 406, 506, 606, 613, 706, 713) configured to allow air to exit through the airway passage and resist air entering through the airway.

According to another aspect that incorporates the first aspect and optionally one or more of the other prior aspects, wherein the at least one valve includes a valve (141, 151, 206, 306, 506, 606, 706) on a distal end of the airway expander.

According to another aspect that incorporates the first aspect and optionally one or more of the other prior aspects, wherein the at least one valve includes a valve (313, 613, 713) on a proximal end of the airway expander.

According to another aspect that incorporates the first aspect and optionally one or more of the other prior aspects, wherein the at least one valve includes a valve (406) positioned in a middle section of the airway expander.

According to another aspect that incorporates the first aspect and optionally one or more of the other prior aspects, the outer dimension of the cutting device in the expanded mode is within a range of: 4.5 mm to 8 mm.

According to another aspect that incorporates the first aspect and optionally one or more of the other prior aspects, wherein the outer dimension of the cutting device in the expanded mode is within a range of: 5.5 mm to 10 mm.

According to another aspect that incorporates the first aspect and optionally one or more of the other prior aspects, wherein the outer dimension of the airway expander in the expended mode is within a range of: 4.5 mm to 8 mm.

According to another aspect that incorporates the first aspect any may incorporate one or more of the previous aspects, wherein the outer dimension of the airway expander in the expended mode is within a range of: 5.5 mm to 10 mm.

According to another aspect that incorporates the first aspect any may incorporate one or more of the previous aspects, wherein the outer dimension of the airway expander is configured to be in a range of 1.5 to 2.5 times greater than an interior outer dimension of the airway prior to expansion.

According to another aspect that incorporates the first aspect any may incorporate one or more of the previous aspects, wherein the outer dimension of the airway expander is configured to be 1 to 10 mm greater than the outer dimension of the airway prior to expansion.

According to another aspect that incorporates the first aspect and optionally one or more of the other prior aspects, the airway expander includes an expandable channel divider (211, 311, 511, 611, 711) longitudinally bifurcating an air passage defined by the airway expander in the expanded mode.

According to another aspect that incorporates the first aspect and optionally one or more of the other prior aspects, wherein the outer dimension of the cutting device in the expanded mode and the outer dimension of the airway expander in the expended mode are each within one or more ranges of: 4.5 mm to 8 mm; and 5.5 mm to 10 mm.

According to another aspect that incorporates the first aspect and optionally one or more of the other prior aspects, wherein the outer dimension of the airway expander is configured to be in a range of 1.5 to 2.5 times greater than an interior outer dimension of the airway prior to expansion or 1 to 10 mm greater than the outer dimension of the airway prior to expansion in the expanded mode of the airway expander.

According to another aspect that incorporates the first aspect and optionally one or more of the other prior aspects, the medical device assembly includes a controller (28) configured to execute the following steps, which may be at least automated:

(i) advance the cutting device (70, 85, 95) into the airway passage (40) to an airway target region (41);

(ii) expand the cutting device from the collapsed mode to the expanded mode while the cutting device is at the airway target region;

(iii) cut the opening (65) in the airway target region during or after the expansion of the cutting device to the expanded mode;

(iv) advance the airway expander (80, 105, 140, 150, 200, 300, 400, 500, 600, 700) through the airway passage (40) to the airway target region (41); and

(v) expand the airway expander at the airway target region (41) to radially displace the airway target region and expand the opening (65).

According to another aspect of the medical device assembly the controller (28) further include executing (vi) collapsing the cutting device from its respective expanded modes to its respective collapsed mode, and (vii) retracting the cutting device from the airway passage.

### BRIEF DESCRIPTION OF THE DRAWINGS

The drawings included herewith are for illustrating various examples of articles, methods, and apparatuses of the present specification and are not intended to limit the scope of what is taught in any way. In the drawings:

FIG. 1A is a schematic illustration of a section of an airway with an undeployed cutting device positioned in the airway.

FIG. 1B is a cross section of FIG. 1A.

FIG. 2A is a schematic illustration of a section of an airway with a deployed cutting device positioned in the airway.

FIG. 2B is a cross section of FIG. 2A.

FIG. 3A is a schematic illustration of a section of an airway with a fenestration cut into the airway.

FIG. 3B is a cross section of FIG. 3A.

FIG. 4A is a schematic illustration of a section of an airway with an undeployed airway expander positioned in the airway.

FIG. 4B is a cross section of FIG. 4A.

FIGS. 5A and 5B are schematic illustrations of a section of an airway with a deployed airway expander positioned in the airway.

FIG. 6A is a schematic illustration of distal region of an undeployed cutting balloon catheter.

FIG. 6B is a cross section of FIG. 6A.

FIG. 6C is a schematic illustration of distal region of a deployed cutting balloon catheter.

FIG. 6D is a cross section of FIG. 6C.

FIG. 7A is a schematic illustration of a cutting catheter.

FIG. 7B is a schematic illustration of a cutting catheter.

FIG. 8A is a schematic illustration of a dilating stent.

FIG. 8B is a schematic illustration of a dilating helical tube.

FIGS. 9A, 9B, 9C and 9D are schematic illustrations of a cutting and stent deploying catheter and method of use.

FIGS. 10 and 11 are schematic illustrations of airway expanders comprising one-way airway valves.

FIG. 12A is a schematic illustration of airway expanders with a dual channel structure, comprising at least one one-way airway.

FIG. 12B is a cross section of FIG. 12A.

FIG. 13 is a schematic illustration of airway expanders with a dual channel structure, comprising at least one one-way airway valve.

FIG. 14 is a schematic illustrations of airway expanders comprising one-way airway valves.

FIG. 15A is a schematic illustrations of airway expanders with a dual channel structure, comprising at least one one-way airway valve.

FIG. 15B is a cross section of FIG. 15A.

FIGS. 16 and 17 are schematic illustrations of airway expanders with a dual channel structure, comprising at least one one-way airway valve in each embodiment.

FIG. 18 is a schematic illustration of a bronchoscope positioning a cutting device and airway expander in an airway passage of a lung.

### DETAILED DESCRIPTION

Systems, devices, and methods are disclosed herein for treating a patient having emphysema by creating an airway bypass with a trans-bronchial device and maintaining patency of the airway bypass with an airway expander. One or more airway bypasses may be created to relive trapped air from a portion of the patient's lung.

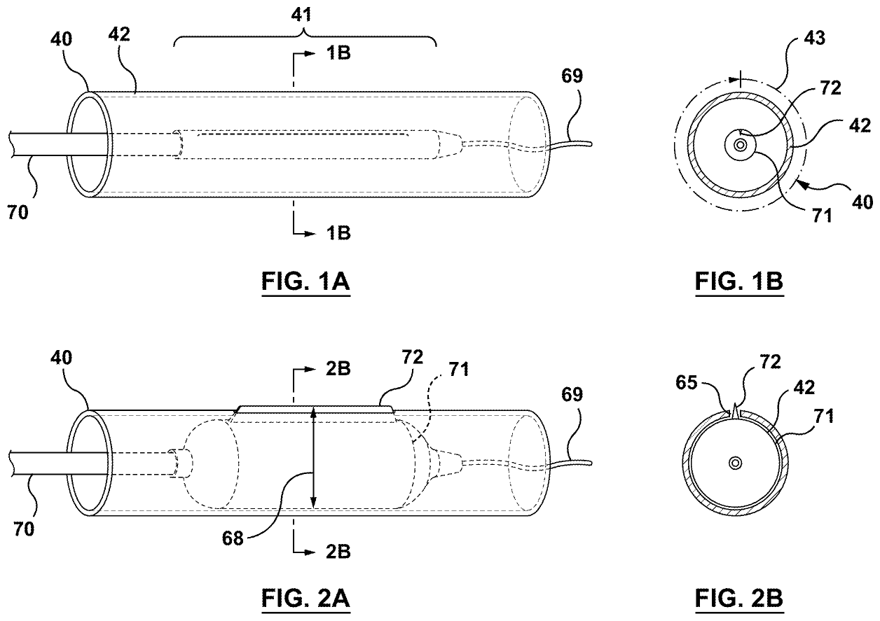

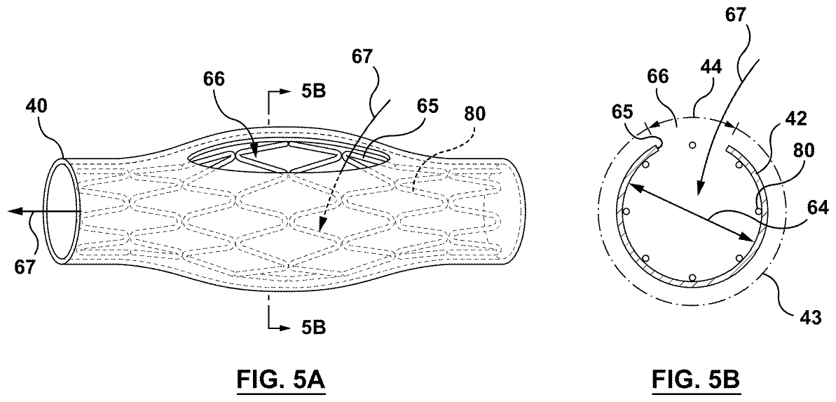

As shown in FIGS. 1A to 5B the airway bypass 66 is created by cutting a fenestration 65 in a target location of an airway with a cutting device. The fenestration may be a longitudinal cut in the airway wall 42 along a longitudinal aspect of the airway, which may be approximately parallel to the axis of the airway or angled (e.g., up to 45 degrees angled to the axis of the airway) and may be substantially straight or curved or have one or more inflection points. Generally, the fenestration 65 is configured to pass through the wall 42 of the airway 40 in a target region 41 such that when the circumference 43 of the airway target region 41 is increased by amount 44 (see FIG. 5B) by an airway expander, shown as a stent 80 in FIGS. 4A to 5B, the fenestration will open (e.g., increase in width and cross-section area) and trapped air will flow 67 from lung parenchyma external to the airway through the fenestration and out of the airway. Importantly the stent is intended to prevent immediate closure of the airway wall by exerting tension on the separated edges of the airway wall fenestration. One or multiple fenestrations may be made in the airway wall. The airway bypass is capable of being implemented in distal airways (e.g., generation 4 or higher) where there is more diseased lung tissue and less risk of bleeding from unintended perforation of a major blood vessel. This may be accomplished by delivery of the cutting device and airway expander over a guidewire under fluoroscopy but can be envisioned to be achieved by other advanced imaging and navigation modalities such as Electromagnetic Navigation Bronchoscopy (ENB) such as the LungGPS™ technology used in the Medtronic superDimension™ navigation system and others.

Targeted airways can be presented as hollow cylinders ranging from 1 mm to 5 mm in inner diameter with a wall thickness ranging, for example, from 0.5 mm to 2 mm.

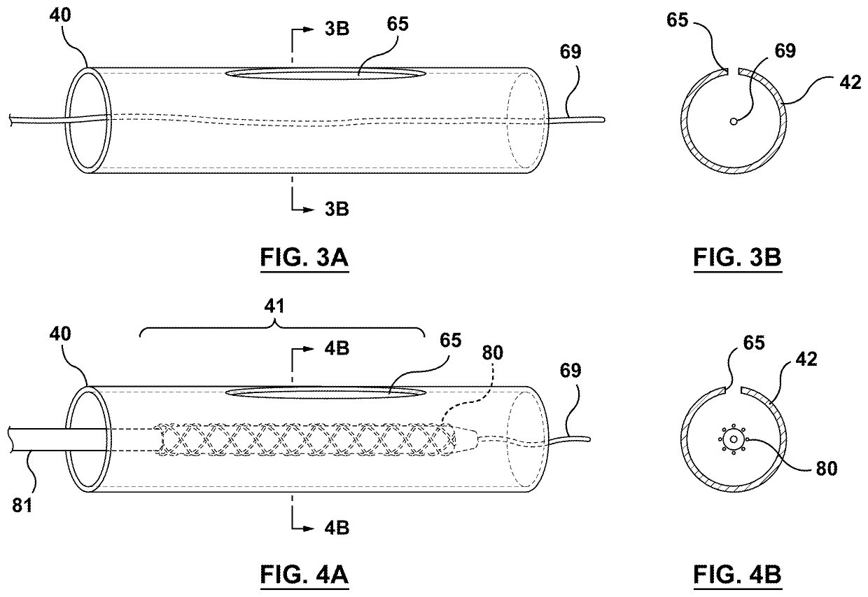

As shown in FIG. 1A to 2B, the fenestration 65 may be cut with a cutting device, for example a cutting balloon catheter 70 delivered over a guidewire 69 as shown, capable of being delivered through the patient's airway 40 to the target region in the airway 41. For example, the cutting device may be delivered at least partially through a bronchoscope (e.g., having a working channel that is 2 mm to 3.5 mm in diameter) to position a cutting tool in the target region of the airway 41 (see FIGS. 1A and 1B). As shown in FIGS. 2A and 2B the cutting device (e.g., cutting balloon 71 mounted to a cutting balloon catheter 70 having a blade 72) may be used to create an incision in the airway wall 42 with a blade. The cutting balloon 71 is expanded to push the blade 72 into and thereby cut a fenestration 65 in the airway wall 42. The expansion of the cutting balloon is an example of an expanded mode of the cutting device. When in the expanded mode, the cutting device has an outer dimension 68 which is the maximum dimension of a cross section of cutting device at the fenestration 65, such as a dimension that is the sum of the diameter of the expanded balloon 71 and the height at which the blade extends from the balloon. Shown in FIGS. 3A and 3B the cutting device may be removed from the airway leaving the guidewire 69 in place. Optionally, in some embodiments wherein the airway expander comprises a cutting tool the cutting tool may be left in place. Optionally, in some embodiments a cut may be made in the airway wall after a step of deploying an airway expander.

The airway expander is a component that is delivered to the inner lumen of the airway at the location of the fenestration and may be for example a stent or a deployable helical tube. In FIGS. 4A and 4B an airway expander is shown in an undeployed configuration on a catheter, for example a stent 80 crimped on a delivery balloon catheter 81 and may have an outer diameter capable of being delivered through a working channel of a bronchoscope (e.g., an outer diameter in its undeployed configuration in a range of 1 mm to 3 mm). The airway expander implant, e.g., stent 80, may be deployed in the target region of the airway 41 to expand the fenestration 65 as shown in in FIGS. 5A and 5B. The airway expander, when in the expanded mode, has an outer dimension 64 which is the maximum dimension of a cross section of the airway expander at the fenestration 65, such as a diameter of a cylindrical airway expander. Compared to prior airway bypass stents such as the Exhale® stent (Broncus Technologies, Mountain View, Calif.) the currently disclosed airway expander (e.g., stent) is placed longitudinally in the airways, not orthogonally. Thus the airway expander can be placed in small diameter, distal airways, unlike prior stents. The airway expander may have a long surface area engaging the airway walls, which could have an improved function of self-anchoring and retaining placement, compared to prior stents that can be expelled by coughing. The airway expander can be expanded repeatedly, for example with a dilation balloon catheter, compared to prior stents that have tendency to close or become blocked over time. For example the stent can be made of stainless steel and expanded by a balloon to a diameter substantially bigger than the airway. For example, as shown in FIG. 5B a circumference 43 of the target region of the airway may be increased by an amount 44 that may be in a range of 1 mm to 5 mm, in a range of 1 mm to 3 mm, in a range of 10% to 30% of circumference 43.

When patient breathes or coughs the airway expander can expand and contract in both longitudinal and circumferential dimensions supporting natural airway motion. For example, an airway expander may have a structure or material that can be elastically deformed under stress of the lung's motion and return to its unstressed state when the stress is removed. It is also an important objective of the invention to allow transport of mucus and secretions by the airway. Thus, it's desired to minimize the contact area of the stent while preserving its stiffness and ability to sustain the airway in the expanded or dilated configuration.

It is anticipated that the lung will attempt to close fenestrations over time through multiple physiologic mechanisms including normal scarring, tissue growth and granulation in response to inflammation and irritation of tissue by the implant. The airway expander can be expanded multiple times, reopening the fenestration, with a dilation balloon if desired. For example, if the fenestration becomes closed or blocked or if the airway expander decreases in circumference over time re-expanding it can reopen the fenestration without having to make a new fenestration.

A cutting device 70, 85, and 95 may be a cutting balloon catheter that has an undeployed configuration having an outer diameter 73 capable of being delivered through a working channel of a bronchoscope (e.g., having a diameter in a range of 1 mm to 3 mm) and over a guidewire (e.g., having a guidewire lumen 74). Cutting balloons have been previously used to prevent restenosis in coronary arteries after cutting balloon angioplasty (CBA) for example U.S. Pat. No. 5,196,024, which is incorporated by reference, but not in an airway.

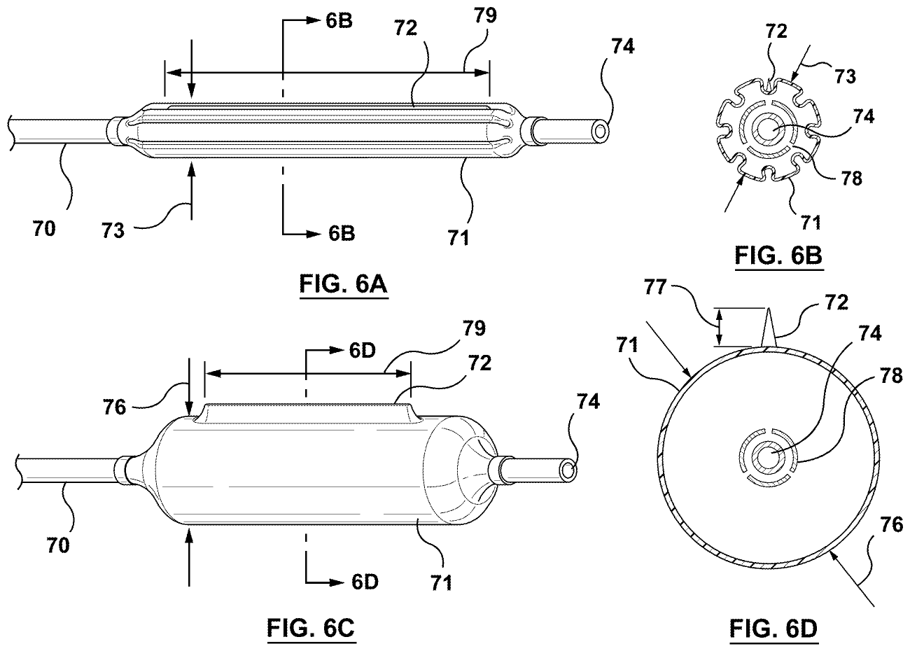

As shown in FIGS. 6A to 6D a cutting balloon catheter 70 may comprise a non-compliant balloon 71 having a length 79 in a range of 10 to 30 mm with at least one micro-blade 72. The cutting balloon 71 may comprise for example up to 4 microblades spaced around the circumference of the balloon. The expanded balloon diameter 76 may be 2 to 10 mm to treat airways that are 1 to 5 mm in diameter. The blade(s) 72 is mounted longitudinally on the balloon 71 and has a height 77 in a range of 0.25 mm to 2 mm, which is sufficient to pass through the airway wall but not much deeper. Alternatively, the height 77 of the blade(s), which is the region of the blade protruding from the balloon, may be sufficient to cut into the airway wall but not all the way through it, for example about 90% through the wall, which may reduce a risk of cutting a blood vessel exterior to the wall and yet allow the partial cut to be pulled open when the airway is dilated. The blade length may be in a range of about 2 mm to 25 mm. The blade length is measured with respect to the longest length of a portion of the blade extending out of the balloon. The length is most likely to be parallel to a longitudinal axis of the balloon. The blade(s) may be contained within a fold of the balloon 71 when the balloon is in its undeployed configuration. When the balloon 71 is inflated by injecting fluid through a lumen of the catheter and through inflation ports 78 within the balloon, the blade(s) is pushed out of the balloon fold and pressed into and optionally through the airway wall. The cutting can be performed with multiple inflations that increasing the balloon pressure, usually up to 2 to 12 atm. Wall thickness to outer diameter ratio (T:D) of sub segmental airways is known to be in a range of 0.1 to 0.35. Thus a balloon to cut a 5 mm diameter airway should have a blade with a height 77 in a range of 0.5 mm to 1.75 mm in order to pass through or significantly through the airway wall. When the airway is dilated and wall tissue is stretched it can be expected to be thinner and a shorter blade (e.g. 1 mm) may succeed in cutting a slit through the 5 mm airway wall. Multiple cutting balloon catheters may be provided with a system or in a kit that are configured for various airway diameter ranges.

It is appreciated that the cutting balloon is an example of a cutting device mounted on a catheter. A cutting device may have an alternative configuration for example it can use electrocautery, a cutting blade deployed from a sheath or other cutting device capable of creating a slit in an airway. Importantly the cutting device is used only during the endobronchial procedure and not implanted.

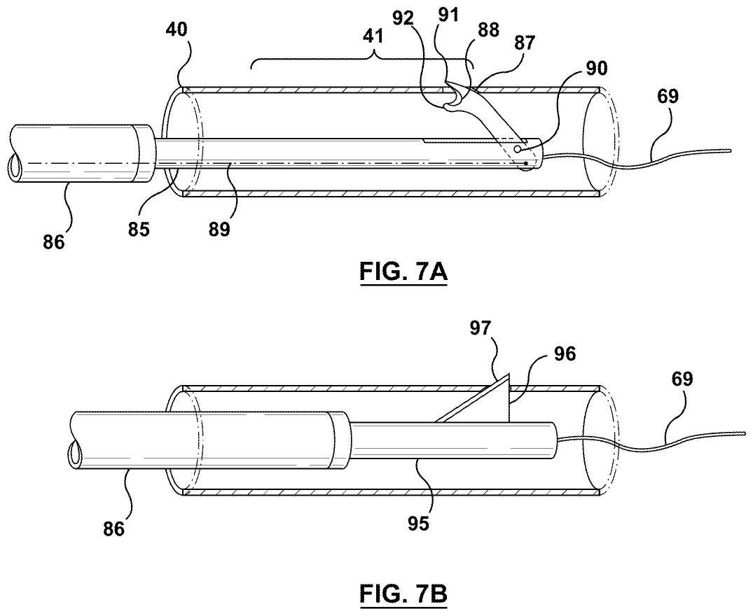

An alternative embodiment of a cutting device is shown in FIG. 7A. A cutting catheter 85 is shown delivered through an optional sheath and over a guidewire 69 into a targeted region of an airway 41. Ability to deliver the cutting catheter over a guidewire facilitates delivery to smaller high generation airways, for example airways having diameters less than 5 mm, and replacement of the cutting catheter with an airway expander delivery catheter to the target region 41. The precise positioning of the implant can be visualized using X-ray during the procedure and is appreciated. The cutting catheter may have a guidewire lumen, which may be positioned off the central axis of the cutting catheter shaft to provide more room for mechanical parts. The cutting catheter 85 comprises a deployable arm 87 having a cutting blade surface 88. The deployable arm 87 may be deployed for example by pulling a pull wire 89 that extends through the catheter and is attached to the arm 87 on a moment arm opposite a pivot hinge 90. A spring, for example a torsion spring mounted about the pivot hinge 90 (not shown), may cause the arm 87 to retract when the pull wire 89 is released. As shown in FIG. 7A the cutting blade surface 88 may comprise a piercing tip 91 for piercing through the airway wall, a crescent shaped blade 88 and a blade guide 92 that guides the blade 88 through the airway wall and prohibits it from extending beyond the airway wall.

Another embodiment of a cutting device is shown in FIG. 7B. In this embodiment, a cutting catheter 95 is delivered through a sheath 86 and a deployable blade 96 is deployed by a spring when a delivery sheath 86 is retracted. The blade 96 has a cutting edge 97 oriented toward the proximal end of the catheter 95.

In these embodiments the blade 88 is oriented toward the proximal end of the catheter and functions to cut the airway by pulling the catheter in the direction of the proximal end. Alternatively, a blade may be oriented toward the distal end and a cutting motion may comprise pushing the catheter. Other motions may alternatively be used to create a fenestration for example pulling while torqueing the cutting catheter 85 may create a helical fenestration. Optionally, this function can be augmented by rifling guiding grooves in the guiding catheter or the design of the handle to precisely create a helical fenestration.

Depth markers may be marked on any embodiment of a cutting catheter shaft that may be visually aligned with an indicator at its proximal region external to the patient to indicate how much the cutting catheter is moved when cutting. For example, depth markers may be marked every 1 mm with a distinct (e.g., longer marker or different color marker) to indicate every 5 mm and the depth markers may be visually aligned with a proximal opening of a bronchoscope working channel or a sheath. A radiopaque marker may be positioned on the distal end of the cutting catheter and a physician may use the marker to see where the target region is, where the cut is made in the airway wall, and how long the cut is.

Following the creation of a fenestration the circumference of the target region of the airway may be expanded, for example with a dilating balloon, then an airway expander such as a stent or helical tube may be implanted in the target region. Alternatively, the airway may be dilated as the airway expander implant is implanted. Alternatively, a fenestration may be made as an airway expander is expanded in an airway.

Dilation and stent deployment can be used with an appropriately sized balloon at high pressure (e.g. 5 to 14 ATM). It is anticipated that distal airways can be dilated more than 20% since they are more flexible than proximal ones where more cartilage is present. It is also anticipated that an airway expander implant can be dilated several times over the course of the progressive emphysema disease.

In one embodiment an airway expander implant is a stent. Stent design can be such that dilation can be repeated with increasingly larger balloons to achieve additional spreading of the fenestration. For each inflated balloon diameter the stent may have a predetermined end-dilation diameter. Many designs of stents exist that allow sequential dilation to different diameters until maximum diameter is reached. Stent in this case may be made out of a stainless steel tube and crimped on the balloon in the delivery configuration.

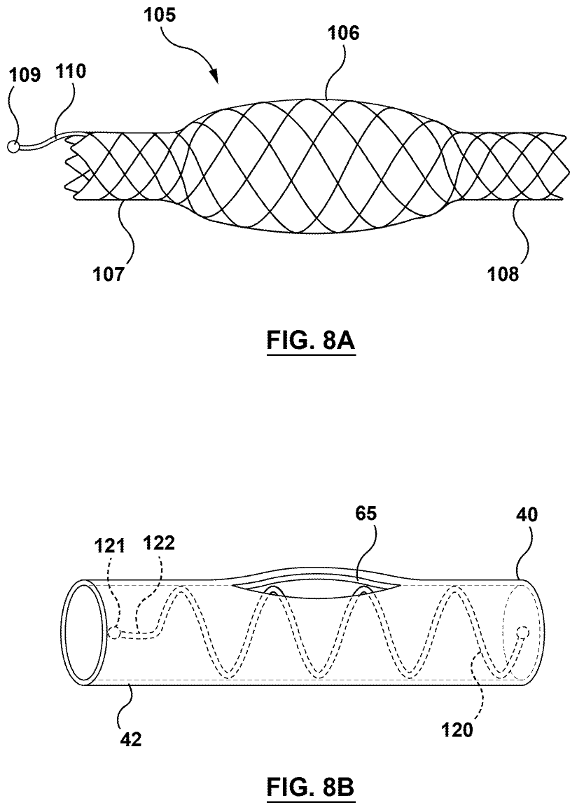

A dilating stent may be cylindrical or alternatively as shown in FIG. 8A a stent, in its expanded state, may comprise a middle section 106 that has a greater circumference than proximal 107 and distal 108 sections. For example, the airway expander in it's maximum open state may have an outer diameter greater than the target location in the airway, optionally in a range of 1.5 to 2.5 times greater, optionally 1 to 10 mm greater. The stent in its minimum closed state may have an outer diameter in a range of 1 to 3 mm. The stent comprises a middle region having a circumference that is larger than the circumference of the airway in it's expanded state, optionally 1 to 5 mm larger, 1 to 3 mm larger, or 10% to 30% of the circumference of the airway. The middle section 106 may have a length approximately the length of the fenestration, for example in a range of 2 mm to 25 mm. The proximal 107 and distal 108 sections may have a diameter to fit the targeted region of the airway, for example in a range of 1 mm to 5 mm, and may facilitate anchoring the stent in the targeted region of the airway. Optionally, the distal section 108 may be tapered or have a smaller diameter than the proximal section 107 to fit airways that taper toward the distal end.

Optionally the stent may be a drug eluting stent that slowly delivers a drug to the tissue to decrease a risk of infection or tissue growing over the fenestration.

Optionally, an airway dilating stent may comprise a valve such as a membrane flap that covers the stents circumference and allows air to flow from lung parenchyma out of the fenestration and out of the lung but not the other direction.

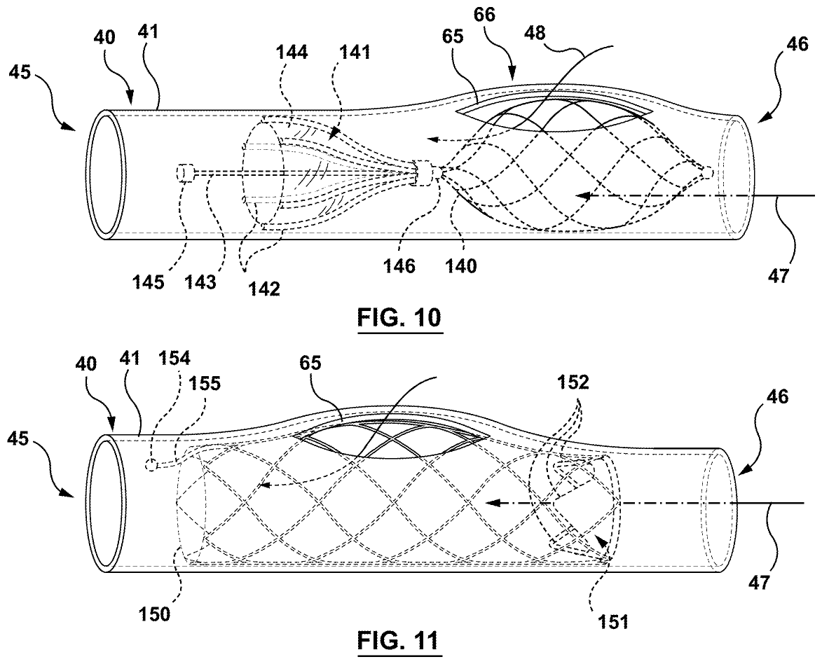

Optionally, as shown in FIGS. 10 and 11 an airway expander (e.g. stent or helical tube) may comprise a one-way valve that allows air to flow one direction through the center lumen of the airway expander but impedes or stops air from flowing the other direction. The distal or proximal portion of the stent may incorporate a valve allowing deflation but not reflation of the distal segment of the lung supplied by the airway. Such a valve can force the lung distal of the fenestration to collapse further improving elastic recoil and reducing hyperinflation of the lung. This combination of a stented fenestration and endobronchial one-way valve may add independent benefit to patients with collateral ventilation that currently benefit less from valve implantation alone.

Valves to facilitate one-way motion of air out of the targeted section of the lung leading to collapse of that section have been proposed before, for example, U.S. Pat. No. 6,258,100 which is incorporated by reference, that help to reduce excessive lung volume in emphysematous lungs. Such prior valves have a well-known limitation of the affected lung section being refilled with air by natural lung porosity that bypasses the airways. The balance of air into and air out of the targeted area is dynamic. It is logical that if the rate of evacuation exceeds the rate of replenishment of the alveolar space, the space will collapse. Currently known valves rely on diseased small airways to evacuate air through the one-way valve. These small airways may be too resistive and collapsible to reliably remove air at a rate faster than it is replenished by porosity of the lung parenchyma, the phenomenon known as collateral ventilation. The placement of endobronchial valves has caused benefit in some patients that have had a significant improvement, responses have been inconsistent because collateral ventilation prevents lobar atelectasis. Lobar atelectasis is a direct consequence of the lung collapse. If it does not occur, valves are generally ineffective and often removed.

We propose to improve the rate of atelectasis counterintuitively by increasing collateral ventilation by creating a targeted airway bypass in the targeted area of the lung. The targeted bypass is intended to deflate the alveolar space at the rate that exceeds replenishing of the targeted area parenchyma from other lobes of the lung through incomplete interlobar fissures. Lung fissures are a double-fold of visceral pleura that either completely or incompletely folds inwards and encloses lung parenchyma to form the lung lobes.

FIG. 10 shows a section of an airway 40 with a fenestration 65 held in place by an expander, in this case a stent 140. The airway 40 has a proximal end 45 and distal end 46, the proximal end leading out of the lung toward the trachea and the distal end leading deeper into the lung toward higher generation airways. In this embodiment a one-way valve 141 is connected to the stent 140 and configured to be positioned on the proximal side of the stent. The valve 141 comprises multiple elastic arms 142 that are connected to a central rod 143 (e.g., crimped) at their distal end and flare outward to contact the airway wall 41 spaced around the circumference of the wall. A flexible membrane 144 is connected to the multiple elastic arms 142 such that when exiting airflow 47 applies a pressure gradient on the valve 141 the membrane 144 deforms to release the exiting airflow 47 toward the proximal end 45 of the airway; when the pressure gradient is reversed (e.g., during inhalation) the valve membrane 144 is pressed against the airway wall 41 sealing the airway and resisting airflow toward the distal end 46 of the airway. The central rod 143 comprises a grasping protrusion 145 to be used for delivery or removal of the device. The central rod 143 (e.g., distal end of the rod 143) may be connected to a stent 140. Optionally the connection 146 between the valve 141 and stent 140 may be articulating to facilitate placement in a curved airway and delivery through bends. The stent 140 is positioned adjacent the fenestration 65 to increase the diameter of the airway to open the fenestration and allow trapped air 48 to exit from surrounding parenchyma into the airway 40 and out of the lung. The device may be delivered by pushing it out of a sheath that constrains the stent 140 and valve 141 to a delivery configuration for example having a maximum diameter in a range of 1.5 mm to 5 mm. The stent 140 may be made from an elastic or superelastic material (e.g. Nitinol, spring steel). When the sheath is retracted the elastic arms 142 deploy toward their preformed shape and the wires of the sent 140 also deploy toward their preformed shape. The preformed shapes of the valve 141 and stent 140 may have a maximum diameter (outer dimension) slightly larger than the targeted airway so they exert and outwardly expanding force on the airway wall 41. For example, a valve-stent device configured to be implanted in an airway having an inner diameter in a range of 2 mm to 3 mm may have a maximum expanded diameter (outer dimension) in a range of 3.5 mm to 6 mm; a device configured to be implanted in an airway having an inner diameter in a range of 3 mm to 4 mm may have a maximum expanded diameter (outer dimension) in a range of 4.5 mm to 8 mm; a device configured to be implanted in an airway having an inner diameter in a range of 4 mm to 5 mm may have a maximum expanded diameter (outer dimenson) in a range of 5.5 mm to 10 mm. Optionally, the stent 140 may have a maximum expanded diameter (outer dimension) that is larger than that of the valve 141 since its function is to exert a force on the airway sufficient to expand its diameter and open the fenestration 65.

An alternative embodiment of an airway expander connected to a one-way valve is shown in FIG. 11 wherein the valve 151 is positioned at the distal end of the stent 150, which is meant to be placed in an airway 40 with the valve 151 oriented toward the distal end 46 of the airway. In this configuration the stent 150 may be deployed with a dilation balloon and may be delivered over a guidewire. The stent 150 may be made from stainless steel. The one-way valve 151 may be made from a membrane (e.g., PTFE, silicone) connected to a superelastic (e.g., Nitinol) wire frame to form leaflets. The leaflets 152 are configured to open when air pressure is greater on the distal end of the airway 46 allowing exiting airflow 47 to escape out of the lung and when the pressure gradient is reversed (e.g., during inhalation) the leaflets close against each other resisting airflow from the proximal end 45 of the airway 40. As shown the valve 151 is a tricuspid valve having three leaflets 152 however other configurations such as a bicuspid valve can be envisioned. The wire frame provides structure to the flexible membrane to maintain the shape of the leaflets 152 and stop them from collapsing when the valve is closed. The valve 151 is connected to the distal end of the stent 150 (e.g., the membrane may be stitched on the stent scaffold, or the wire frame may be incorporated as stent splines.) Optionally, the valve 151 may be connected to the proximal end of the stent 150. Optionally, the stent may comprise a grasping protrusion connected to its proximal end for example wherein the grasping protrusion is comprised of a ball-shape 154 or disc-shape connected to the stent 150 with a neck 155, the neck 155 having a curvature that holds the ball-shape 154 or disc-shape away from the airway wall 41.

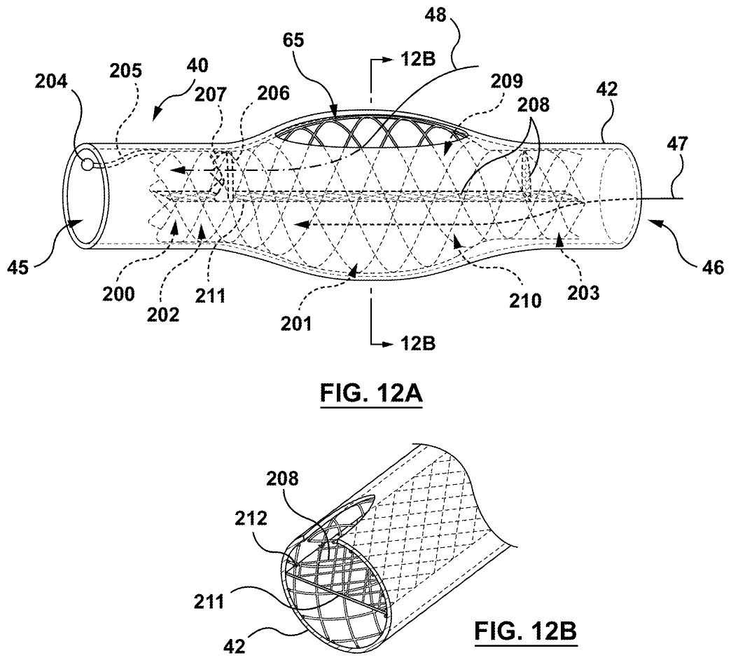

An alternative embodiment of an airway expander is shown in FIG. 12A. The expander may be a stent 200 made from an elastic or superelastic material (e.g. Nitinol, spring steel). The stent 200 may comprise a middle section 201 that has a greater circumference than proximal 202 and distal 203 sections in its expanded state. The stent has a dual channel structure, which means that the stent channel is divided into two channels by a center wall or channel divider 211 longitudinal to the stent channel. The channel divider 211 may also be made from elastic wire (e.g., nitinol wire), shown in FIG. 12B. In the expanded and implanted state, one of the channels, Channel 1 (209), is facing and expanding the cut fenestration 65 on the airway wall 42, while the other channel, Channel 2 (210) is in the same section of the airway but not in direct fluid communication with the fenestration (e.g., on the further side of the fenestration). At least, the channel divider 211 and the wall of Channel 1 is covered by a membrane 208, so that there is no direct air communication between Channel 1 (209) and Channel 2 (210), and there is no air leaking through the wall of Channel 1 to the airway 40. There is a one-way valve 206 fixed to the proximal side of Channel 1. The valve 206 is shaped and sized to fit the cross section of Channel 1. The one-way valve 206 may be made from a membrane (e.g., PTFE, silicone) connected to a superelastic (e.g., Nitinol) wire frame to form leaflets 207. The leaflets 207 are configured to open when air pressure is less on the proximal side of the airway 45 (e.g., during exhalation), allowing trapped air flow 48 to escape out of the lung. When the pressure gradient is reversed (e.g., during inhalation), the leaflets 207 close against each other, resisting airflow from the proximal end 45 of the airway 40 into Channel 1 or the diseased area. As shown the valve 206 is a tricuspid valve having three leaflets 207, however other configurations such as a bicuspid valve or duckbill valve can be envisioned. The wire frame provides structure to the flexible membrane to maintain the shape of the leaflets 207 and stop them from collapsing when the valve is closed. The distal side of Channel 1 may be sealed by a membrane 208 as well. Channel 2 (210) has complete patency, which allows the healthy airway or airways distal to the stent to function normally. The device may be delivered by pushing it out of a sheath that constrains the stent 200 and its valve 206 to a delivery configuration, for example having a maximum diameter (outer dimension) in a range of 1.5 mm to 5 mm. Optionally, the stent 200 may be delivered over a guidewire, which may run through Channel 2 (210). The protrusion 204 and its arm/neck 205 can be located and bent from the circumference of the stent, or extended from the channel divider 211 plane for manipulating, adjustment and retrieving operations.

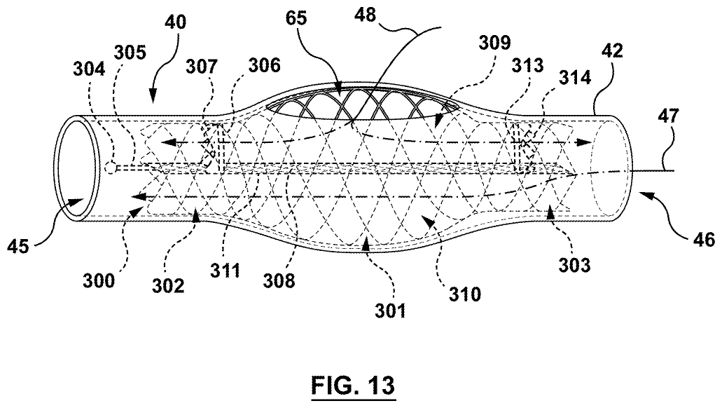

FIG. 13 provides another embodiment of an airway expander. The expander is also a dual channel structure with a channel divider 311, but it comprises two one-way valves 306, 313 in its Channel 1 (309). At least, the channel divider 311 and the wall of Channel 1 is covered by a membrane 308, so that there is no direct air communication between Channel 1 (309) and Channel 2 (310), and there is no air leaking through the wall of Channel 1 to the airway 40. One of the one-way valves 306 is fixed to the proximal side of Channel 1, while the other valve 313 is fixed to the distal side of Channel 1. The leaflets 307, 314 of the two valves are configured to open when air pressure is less in the target airway 40, 45, 46, allowing trapped air flow 48 to escape out from the diseased area, and when the pressure gradient is reversed, the leaflets 307, 314 close against each other, resisting airflow from the proximal end 45 or the distal end 46 of the airway 40 into Channel 1 or the diseased area. Channel 2 (310) has complete patency, which allows the healthy airway or airways distal to the stent to function normally. The device may be delivered by pushing it out of a sheath that constrains the stent 300 and its valves 306, 313 to a delivery configuration, for example having a maximum diameter (outer dimension) in a range of 1.5 mm to 5 mm. Optionally, the stent 300 may be delivered over a guidewire, which may run through Channel 2 (310). The protrusion 304 and its arm/neck 305 can be located and bent from the circumference of the stent, or extended from the channel divider 311 plane for manipulating, adjustment and retrieving operations.

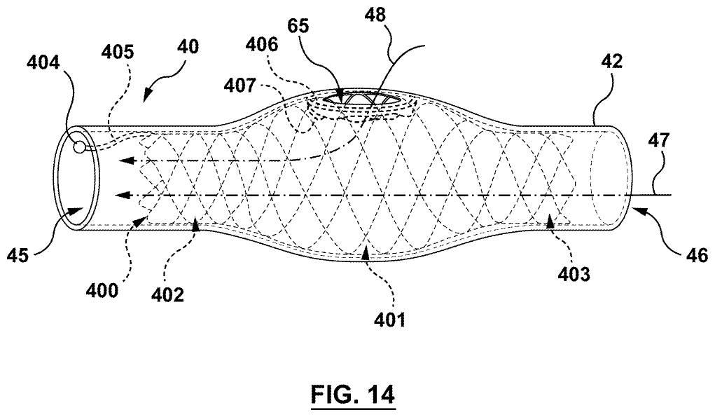

Another embodiment of an airway expander is shown in FIG. 14. It is a single channel stent 400. In the deployed state, the one-way valve 406 is positioned in the dome of the middle section of the stent 400. Since the fenestration 65 on the target airway wall may optionally be a small cut or a short slit, created via piercing, puncturing or even snipping or burning, which is not necessarily as large as or as long as the slits cut in the other embodiments, the middle section of the stent 401 doesn't have to be as long as that in the other designs, optionally could have a spherical shape, which is able to expand the small fenestration. The leaflets 407 on the valve 406 are configured to open when air pressure is less on the proximal side of the airway 45 (e.g., during exhalation), allowing trapped airflow 48 to escape out of the lung and when the pressure gradient is reversed (e.g., during inhalation), the leaflets 407 close against each other, resisting airflow from the airway 40 into the diseased area. The device may be delivered by pushing it out of a sheath that constrains the stent 400 and its valve 406 to a delivery configuration, for example having a maximum diameter (outer dimension) in a range of 1.5 mm to 5 mm. Optionally, the stent 400 may be deployed with a dilation balloon and may be delivered over a guidewire. The protrusion 404 and its arm/neck 405 can be located and bent from the circumference of the stent for manipulating, adjustment and retrieving operations.

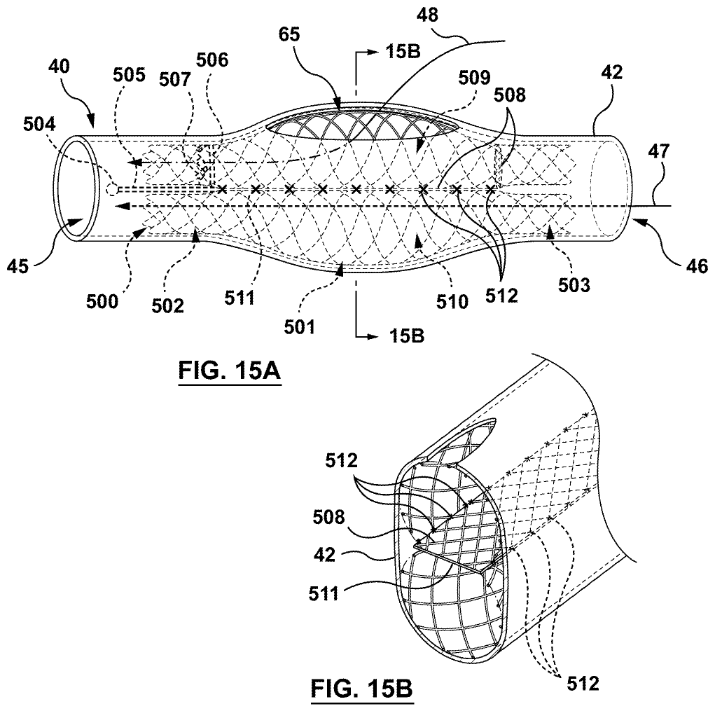

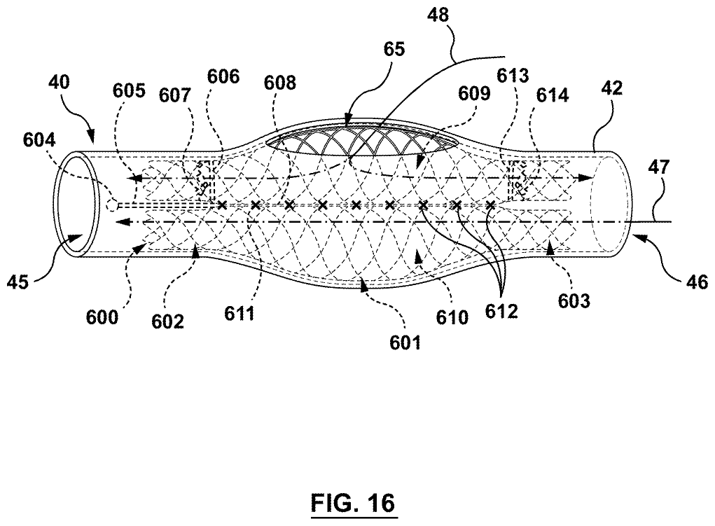

In another embodiment of the airway expander, as shown in FIG. 15A, a dual channel stent 500 is comprised of Channel 1 (509) and Channel 2 (510), which are two substents to the stent 500 and connected to each other at, at least one sequence of, the joining points 512 along the longitudinal direction (Shown in FIG. 15B). The stent 500, or Channel 1 and 2 may comprise a middle section 501 that has a greater circumference than proximal 502 and distal 503 sections in the expanded state. In the expanded and implanted state, one of the channels, Channel 1 (509), is facing and expanding the cut fenestration 65 on the airway wall 42, while the other channel, Channel 2 (510) is on the opposite side in the same airway. At least, the wall of Channel 1 (509) is covered by a membrane 508, so that there is no direct air communication between Channel 1 and Channel 2, and there is no air leaking through the wall of Channel 1 to the airway 40. There is a one-way valve 506 fixed to the proximal side of Channel 1 (509). The valve 506 is shaped and sized to fit the cross section of Channel 1. The one-way valve 506 may be made from a membrane (e.g., PTFE, silicone) connected to a superelastic (e.g., Nitinol) wire frame to form leaflets 507. The leaflets 507 are configured to open when air pressure is less on the proximal side of the airway 45 (e.g., during exhalation), allowing trapped air flow 48 to escape out of the lung, and when the pressure gradient is reversed (e.g., during inhalation), the leaflets 507 close against each other, resisting airflow from the proximal end 45 of the airway 40 into Channel 1 or the diseased area. As shown, the valve 506 is a tricuspid valve having three leaflets 507, however other configurations such as a bicuspid valve, a duckbill valve, an overlapping membrane flap valve or others can be envisioned. The wire frame may provide structure to the flexible membrane to maintain the shape of the leaflets 507 and stop them from collapsing when the valve is closed. The distal side of Channel 1 is sealed by a membrane 508, or alternatively have a one-way valve 613 fixed to the distal side of Channel 1 (609) allowing air to flow out Channel 1 toward a distal portion of the airway 46, as shown in another embodiment in FIG. 16. Channel 2 (510, 610) has complete patency, which allows the healthy airway or airways distal to the stent to function normally. The device may be delivered by pushing it out of a sheath that constrains the stent 500, 600 and its valve 506, 606, 613 to a delivery configuration, for example having a maximum diameter (outer dimension) in a range of 1.5 mm to 5 mm. Optionally, the stent 500, 600 may be delivered over a guidewire, which may run through Channel 2 (510, 610). The protrusion 504, 604 and its arm/neck 505, 605 can be located and bent from the circumference of the stent, Channel 1, Channel 2, or extended from the channel connection 511, 611 plane for manipulating, adjustment and retrieving operations.

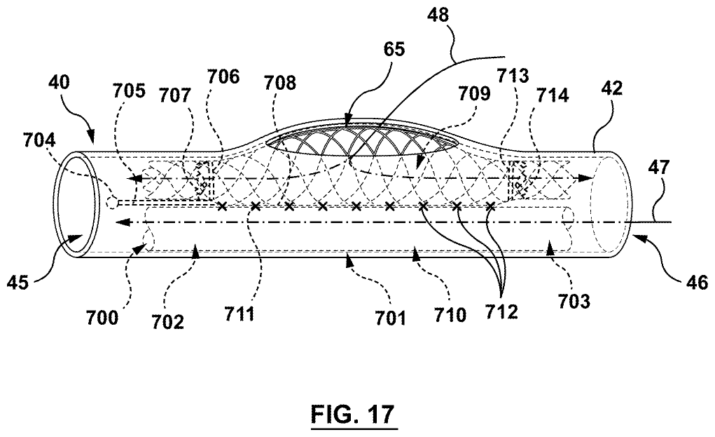

FIG. 17 provides another embodiment of an airway expander. The two channels of the expander are connected at the joining points 712 along the longitudinal direction. Channel 2 (710) may be a straight tube or catheter, which doesn't have to be a woven stent. The length of the tube or Channel 2 is at least the same length of the middle section of Channel 1. Channel 1 (709) comprises two one-way valves 706, 713. At least, the wall of Channel 1 is covered by a membrane 708, so that there is no direct air communication between Channel 1 (709) and Channel 2 (710), and there is no air leaking through the wall of Channel 1 to the airway 40. One of the one-way valves 706 is fixed to the proximal side of Channel 1, while the other valve 713 is fixed to the distal side of Channel 1. The leaflets 707, 714 of the two valves are configured to open when air pressure is less in the target airway 40, 45, 46, than in targeted lung parenchyma exterior to the airway allowing trapped air flow 48 to escape out from the diseased area, and when the pressure gradient is reversed, the leaflets 707, 714 close against each other, resisting airflow from the proximal end 45 or the distal end 46 of the airway 40 into Channel 1 or the diseased area. Channel 2 (710) has complete patency, which allows the healthy airway or airways distal to the stent to function normally. The device may be delivered by pushing it out of a sheath that constrains the stent 700 and its valves 706, 713 to a delivery configuration, for example having a maximum diameter (outer dimension) in a range of 1.5 mm to 5 mm. Optionally, the stent 700 may be delivered over a guidewire, which may run through Channel 2 (710). The protrusion 704 and its arm/neck 705 can be located and bent from the circumference of the stent, Channel 1, Channel 2, or extended from the channel connection 711 plane for manipulating, adjustment and retrieving operations.

Optionally, the airway expander may be made from bioabsorbable material or partially made from bio absorbable material that over time degrades and can be absorbed by the body. Optionally, the implant altogether is removable by pulling it out with a grasping device deployed by the bronchoscope. For example a forceps tool delivered through bronchoscope may be used to grasp a grasping protrusion 109, 204, 304, 404, 504, 604, 704 that may be a ball-shape attached to the stent 105, 140, 150, 200, 300, 400, 500, 600, 700 with a wire neck 110, 205, 305, 405, 505, 605, 705 that holds the ball shape off the airway wall so it is easier to grasp.

An alternative embodiment of an airway expander may be a helical tube 120 as shown in FIG. 8B. The helical tube may be made for example of Nitinol with a preformed helical shape (outer dimension) which may be inserted through a delivery sheath in a constrained state and elastically deploy into an expanded state within the airway. Such designs may have advantage of less interference with mucus transport and more elastic motion with the moving lung. Ends of the helix may be equipped with a grasping protrusion attachment protruding into the airway to facilitate removal. The grasping protrusion may be a ball-shape 121 on an arm 122 bent to position the ball-shape near an axis of the helix so the ball-shape is positioned in the airway 40 away from the airway wall 42 when the airway expander is deployed in the airway so it is easily grasped for example by an endoscopic forceps tool.

It can be envisioned that the stent deployment balloon can be also equipped with cutting blades to combine cutting and dilating airways into one operation. For example, an airway dilating stent may be crimped on an expanding balloon that comprises a deployable cutting blade configured to extend between wires of the stent and into the airway wall. The deployable cutting blades may be small enough to pass through single open cells of the stent creating multiple unconnected cuts. The stent and expanding balloon may be pulled together with the blades protruding into the airway wall to slide the blades and join the discrete cuts.

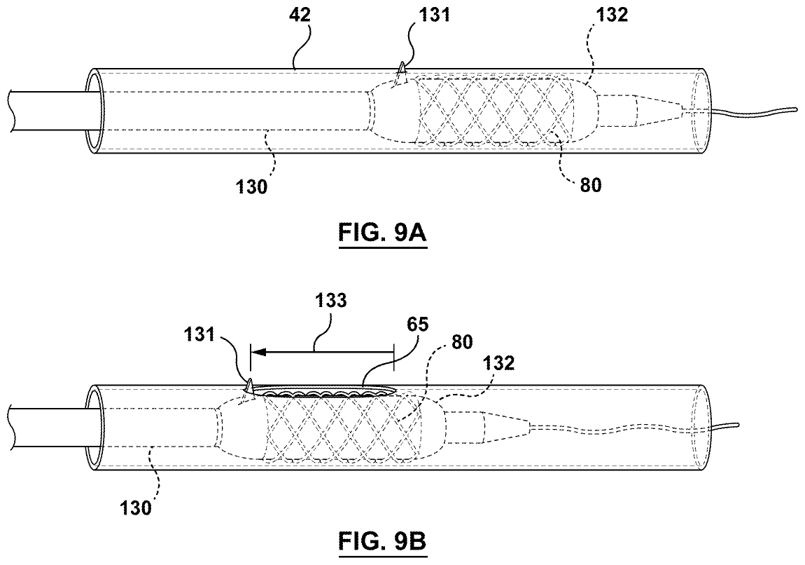

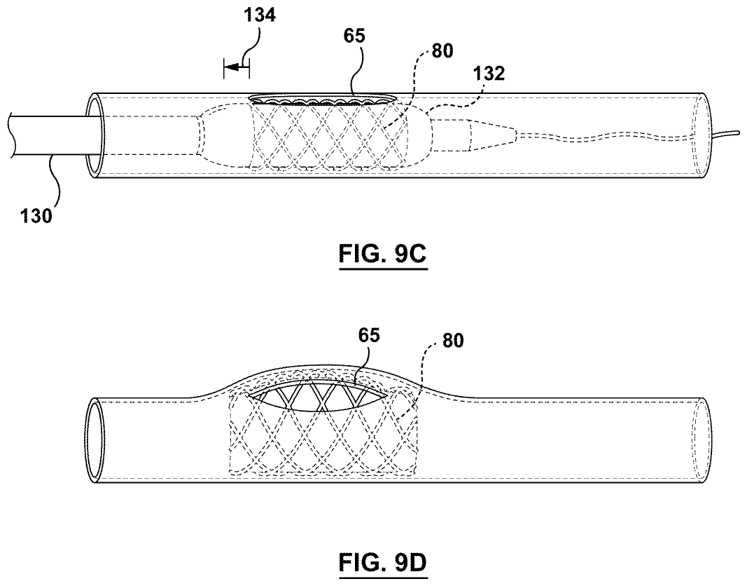

Alternatively, as shown in FIGS. 9A to 9D a catheter 130 for creating an airway bypass may be configured to both create a fenestration 65 and implant a stent 80, 150, 200, 300, 400, 500, 600, 700 by deploying at least one short blade 131 from the catheter for example by partially deploying a balloon 132, the blade piercing through the airway wall 42 (FIG. 9A). The balloon 132 may have the crimped stent 80, 150, 200, 300, 400, 500, 600, 700 mounted to it. The catheter 130 may be pulled toward a proximal direction a distance corresponding to a desired length 133 of a fenestration 65 to pull the blade 131 through the airway wall (FIG. 9B). The blade 131 may optionally be retracted and the catheter 130 may be pulled toward a proximal direction a distance 134 to center the stent 80, 150, 200, 300, 400, 500, 600, 700 over the fenestration 65 (FIG. 9C). Then the stent may be expanded more to open the fenestration and the balloon catheter 130 may be deflated and removed from the airway leaving the stent in place (FIG. 9D). For the dual channel structure stents 200, 300, 500, 600, 700, further alignment to have one channel of the stent facing towards the fenestration may be performed if necessary.

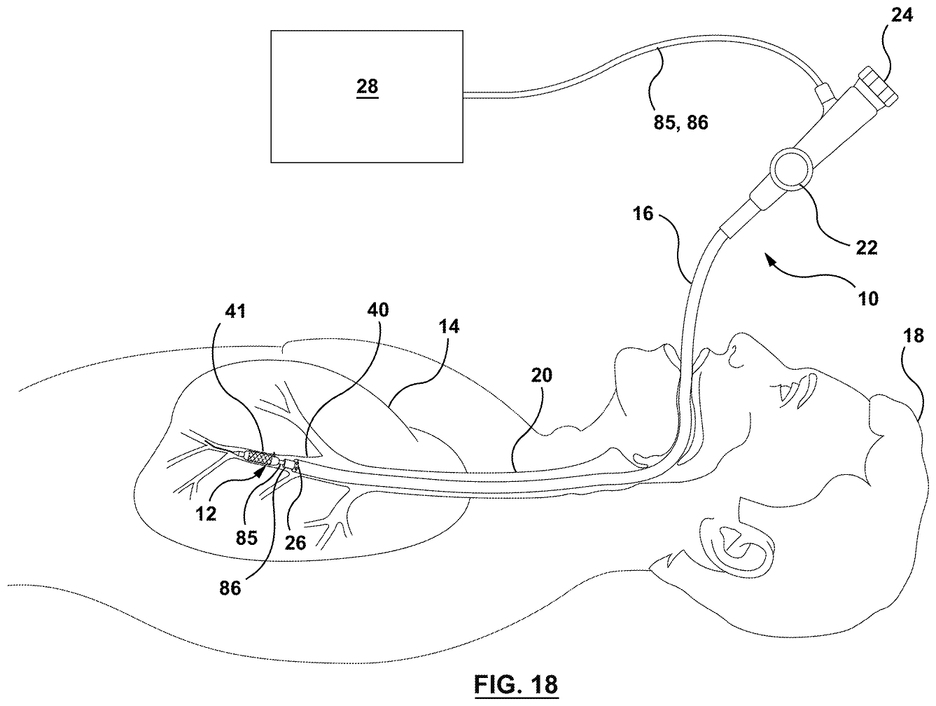

FIG. 18 is a schematic illustration of a bronchoscope 10 positioning an assembly 12 of a cutting device and an airway expander in an airway 40 of a lung 14. A tubular insertion section 16 of the bronchoscope is inserted through the mouth of a patient 18 and maneuvered through the patient's trachea 20 and into the airways of the lung. The tubular insertion section 16 may be maneuvered using controls 22 and a viewer 24, such as a display, at a proximal end of the bronchoscope.

The tubular insertion section 16 includes a working channel which extends from the proximal end region of the bronchoscope to a distal end 26 of the tubular section. A sheath 86 and a catheter 85 within the sheath extend through the working channel and extend out the distal end 26 of the tubular member. Proximal ends of the catheter 85 and sheath 86 extend from the proximal region of the bronchoscope.

A controller 28 is connected to the proximal ends of the catheter 85 and sheath 86. The controller 28 may be configured to position, e.g. advance laterally and rotate, the sheath 86 and catheter 85 through and out from the working channel and into the airway 40. Such configuration may include mechanical gears and motors which move the sheath and catheter. The catheter is movable independently of the sheath to position the assembly 12 of the cutting device and the airway expander at the target region 41 in the airway 40.

The controller 28 may also be configured to expand and contract the cutting balloon and the airway expander. Such configurations may include a fluid source, such as a source of saline, and a pump to pump the liquid through a lumen in the catheter and into the catheter balloon and into a balloon to expand the airway expander.

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

1.-38. (canceled)

39. A method of treating a patient with emphysema comprising:
creating an airway bypass passage in a wall of a target location in an airway of a lung of the patient,
deploying an airway expander in the target location in the airway,
expanding the airway expander to expand the airway bypass passage by expanding the airway diameter.

40. The method of claim 39 wherein the step of creating an airway bypass passage comprises delivering an airway cutting device through the patient's airway to the target location and cutting a fenestration.

41. The method of claim 40 wherein the delivering step comprises delivering through a bronchoscope.

42. The method of claim 40 wherein the delivering step comprises delivering through a sheath.

43. The method of claim 40 wherein the delivering step comprises delivering over a guide wire.

44. The method of claim 39 wherein the airway cutting device is a deployable blade deployed by a balloon, a pull wire, retracting a sheath, or a stent.

45. The method of claim 39 wherein the airway bypass passage is a fenestration with a length in a range of 2 to 25 mm.

46. The method of claim 39 wherein the airway expander is placed longitudinally in the airway.

47. The method of claim 39 wherein the airway expander in its maximum open state has an outer diameter in a range of 1.5 to 2.5 greater than the target location in the airway.

48. The method of claim 39 wherein the airway expander in its minimum closed state has an outer diameter in a range of 1 to 3 mm.

49. The method of claim 39 wherein the airway expander comprises a middle region having a circumference that is larger than the circumference of the airway in its expanded state, optionally 1 to 5 mm larger, 1 to 3 mm larger, or 10% to 30% of the circumference of the airway.

50. The method of claim 39 wherein the airway expander is a stent or a helical wire.

51. The method of claim 39 wherein the airway expander is a stent, which can be a single channel structure or dual channel structure.

52. The method of claim 39 wherein for the dual channel stent, the wall of at least one channel of the stent is covered by membrane.

53. The method of claim 39 wherein the airway expander comprises at least one one-way valve that allows air to expel from the airway bypass passage but not enter it.

54. The method of claim 53 wherein the one-way valve is a membrane.

55. The method of claim 39 wherein the airway expander comprises a one-way valve that allows air to expel from the airway but not enter it.

56. The method of claim 39 further comprising a step of expanding the airway expander a repeated time, optionally comprising delivering a dilating balloon to the airway expander and inflating the dilating balloon to apply pressure to the inside of the airway expander.

57. The method of claim 39 wherein the target location is a generation 4 or higher airway.

58. The method of claim 39 wherein the airway expander and the cutting device are the same device.

59. The method of claim 39 wherein the step of making the airway bypass passage is accomplished during the step of deploying the airway expander.

60. The method of claim 39, wherein the target location is adjacent emphysematous lung parenchyma.

61. The method of claim 39, wherein the target location is adjacent a hyper-inflated portion of lung parenchyma.

62.-81. (canceled)

82. A method of reducing a volume of a target section of a lung by at least collapsing or partially collapsing the target section of the lung, the method comprising steps of:
delivering within an airway leading to the target section an intra-bronchial valve device having an obstructing member supported on a support structure, the obstructing member being configured to preclude air from being inhaled into the target section, while allowing air to be exhaled from the target section, the valve device further comprising at least one airway expander configured to expand the airway diameter, the airway expander configured to bypass natural airways by creating and supporting a fenestrated air passage into lung parenchyma.

83. A method for lung volume reduction comprising steps of:
creating an airway bypass passage between a targeted area of lung parenchyma adjacent to a targeted airway by forming a fenestration in a wall of the targeted airway at a site of the airway bypass passage; and
after the formation of the fenestration in the airway wall, implanting at least one airway expander configured to expand a diameter of the targeted airway at the site of the airway bypass passage;
expanding the fenestration to prevent or or at least delaying closure of the fenestration, wherein the expansion is performed by expanding the at least one airway expander.
